(19) United States
(12) Patent Application Publication
(10) Pub. No.: US 2026/0033724 A1
(43) Pub. Date: Feb. 5, 2026

(54) METHODS AND SYSTEMS FOR INFLUENCING BLOOD FLOW

(71) Applicant: Wallaby Medical, LLC., Laguna Hills, CA (US)

(72) Inventors: Earl Bardsley, Marietta, SC (US); James Lago, Las Vegas, NV (US)

(73) Assignee: Wallaby Medical, LLC., Laguna Hills, CA (US)

(21) Appl. No.: 19/284,586

(22) Filed: Jul. 29, 2025

Related U.S. Application Data
(60) Provisional application No. 63/677,392, filed on Jul. 30, 2024.

(51) Int. Cl. A61B 5/00; A61B 5/02; A61B 17/12

(52) U.S. Cl. CPC A61B 5/0066; A61B 5/0084; A61B 5/02014; A61B 17/12113

(57) ABSTRACT

The present teaching provides device and method of use thereof, for example, for the placement of a medical implant inside the cerebrovasculature. Various embodiments of the present teachings include a medical implant, a delivery system, and an Optical Coherence Tomography (OCT) imaging system. The OCT imaging system is used to reveal the three dimensional (3D) volumetric structure of the vascular network, as well as vessel density and geometric features, blood flow velocity, flow rate, and other data useful for selecting suitable medical implant as well as treatment location. This OCT imaging system is also used to guide the medical implant deployment and post implantation assessment.

### FIELD

The present teaching relates to devices, for example, a medical implant, and methods of use thereof, for example, accurately placing such a device inside the cerebrovasculature. An example of the present teaching relates to the delivery and deployment of a medical implant under the guidance of an Optical Coherence Tomography (OCT) imaging system.

### BACKGROUND

A brain aneurysm is a bulge or ballooning in a blood vessel in the brain. It's also known as a cerebral aneurysm or intracranial aneurysm. This can lead to serious complications, including brain damage and death. The implantation of platinum coils has worked well, particularly in situation where platinum coils fills up the aneurysm entirely, blocks the inflow of blood, results in the formation of a local thrombus, and ultimately a healing process of the aneurysm neck. However, this treatment method is best for aneurysms with narrow necks (inlet for flow from artery). For an aneurysm with a wide neck, coils do not present an optimal therapy for healing of the aneurysm neck.

Flow diversion with stents is a technique used to treat brain aneurysms and involves redirecting blood flow away from the aneurysm and promoting gradual closure of the aneurysm. In practice, a catheter is used to place a mesh tube (a flow diverter) into the blood vessel where the aneurysm is located. The flow diverter effectively diverts blood flow and encourages the aneurysm to thrombose (clot) and finally heal. Eventually, the aneurysm is isolated from the circulation. The flow diverter is placed within the parent artery rather than the aneurysm sac.

While flow diversion is generally effective in redirecting blood flow and promoting aneurysm occlusion, complications can arise during or after the procedure. For example, a flow diverter can inadvertently block small arteries that are branching off the main vessel, potentially leading to ischemic events. In some occasions, periprocedural complications such as a vessel perforation or rupture could occur during the procedure. In other occasions, perianeurysmal edema or distant infarctions could also occur.

The cerebrovasculature, or the network of blood vessels that supply blood to the brain, has several critical and unique characteristics that differentiate it from the vasculature in other organs. Cerebral blood vessels are known for their intricate and often tortuous paths, particularly in cases of conditions like atherosclerosis, hypertension, and certain genetic disorders. Navigating catheters through these winding vessels, especially into distal branches, can be challenging and increase the risk of complications. Obtaining clear visualization of the cerebrovasculature during catheterization using conventional methods, such as Digital Subtraction Angiography (DSA) or standard CT, can be challenging. In complex cases, precise catheter-placement is difficult.

The lack of visualization hinders accurate assessment of stent/flow diverter deployment and arterial wall apposition. Potential complications like in-stent stenosis can also occur. Manufacturers are developing flow diverters with integrated radiopaque materials, like by using Nitinol wire with a platinum core, to improve their visibility during the delivery and placement of a flow diverter. Although a radiopaque material enhances the device visibility, the need of clear mapping of the implantation environment remains unsatisfied. As a result, the risk of blocking small arteries or vessel perforation, remains. In addition, an improved visualization of the intricate network of blood vessels within the brain is crucial for both diagnosing and treating a variety of neurological conditions and these neurological conditions include strokes, aneurysms, and vascular malformations.

### SUMMARY

One aspect of the present teaching provides a medical system for treating a cerebrovascular condition. The medical system comprises a microcatheter, a medical implant, a delivery shaft, and an OCT imaging system. The microcatheter has a longitudinal lumen and a distal end. The medical implant has a proximal end, a distal end, and a central lumen. The medical implant also has a radially collapsed delivery profile to fit inside the longitudinal lumen of the microcatheter. The medical implant also has a radially expanded deployment profile to fit inside the cerebrovasculature. The delivery shaft has a longitudinal lumen and a distal end. The distal end of the delivery shaft detachably engages to a proximal end of the medical implant. The OCT imaging system has an elongated shaft with an optical core and an optical assembly at a distal end of the optical core. The optical assembly attaches to the elongated shaft. The OCT imaging system, the delivery shaft, and the microcatheter extends and retracts independent from one another. The medical system has a first configuration where the medical implant slidably disposes within the longitudinal lumen of the microcatheter; the OCT imaging system slidably disposes within the longitudinal lumen of the delivery shaft and the central lumen of the medical implant; and the optical assembly of the OCT imaging system extends distally beyond the distal end of the microcatheter. The medical system has a second configuration where the medical implant is outside of the microcatheter with the optical assembly of the OCT imaging system extending distally beyond the distal end of the medical implant.

In various embodiment, the medical implant of the preset teaching has a braid structure comprising a plurality of filaments intertwined with each other.

In various embodiment, the medical implant of the preset teaching engages to the delivery shaft when the proximal end of the medical implant remains inside the longitudinal lumen of the microcatheter. In another embodiments, the medical implant of the present teaching detaches from the delivery shaft when the proximal end of the medical implant is outside of the longitudinal lumen of the microcatheter.

In various embodiment, the OCT imaging system of the preset teaching connects optically and mechanically with an interface unit. In another embodiments, the OCT imaging system is configured to collect and transmit image data of the cerebrovasculature to the interface unit.

Another aspect of the present teachings provides a method of placing a medical implant inside a cerebrovasculature. The method comprises advancing an OCT imaging system into a cerebrovasculature, extending a microcatheter over an elongated shaft of the OCT imaging system, placing a distal end of the microcatheter near a treatment location, engaging the medical implant to a delivery shaft, extending the medical implant and the delivery shaft over the elongated shaft of the OCT imaging system; advancing the medical implant through an elongated lumen of the microcatheter to the treatment location, retracting the microcatheter proximally, and allowing the medical implant to expand radially under the guidance of the OCT imaging system.

In various embodiment, method of placing a medical implant inside a cerebrovasculature further includes collecting and assessing real-time image data of the cerebrovasculature at the treatment location by using the OCT imaging system prior to extending the microcatheter to the treatment location.

In various embodiment, method of placing a medical implant inside a cerebrovasculature further includes selecting the medical implant based on the real-time image data of the cerebrovasculature.

In various embodiment, method of placing a medical implant inside a cerebrovasculature further includes selecting a medical implant deployment position based on the real-time image data of the cerebrovasculature.

### BRIEF DESCRIPTION OF THE DRAWINGS

FIG. 1 is a perspective view of an exemplary medical system in accordance with the present teachings.

### DETAILED DESCRIPTION

In one aspect, the present teachings provide a medical system capable of influencing the flow of blood in a vessel. For example, the present teachings provide a medical implant, an implant delivery system, and an intravascular imaging mechanism. In many embodiments, a medical implant of the present teachings is used to seal off blood flow to an aneurysm, arteriovenous malformation, a tumor, or a condition needing thereof. In many embodiments, a delivery system of the present teachings is for introducing the medical implant into blood vessels or hollow organs of a human body. In many embodiments, an intravascular imaging mechanism of the present teachings is to qualitatively map the blood vessel network and/or quantitatively measure the blood flow before and after the deployment of a medical implant.

According to one embodiment of the present teachings, the medical implant is formed by braiding a plurality of filaments into a generally tubular shape. In many embodiments, the medical implant has a proximal end and a distal end. In many embodiments, the braided implant has a radially collapsed elongated profile, for example, being suitable to be positioned inside a microcatheter, and a radially expanded deployed profile, for example, capable of adapting to the vessel diameter when the braided implant is placed at an implantation site. During the delivery process, the medical implant collapses radially, and the collapsed medical implant is advanced distally through a microcatheter. In many embodiments, a delivery system of the present teachings includes a delivery shaft and a microcatheter. In some embodiments, the delivery shaft includes a distal end, where the distal end of the delivery shaft is configured to engage a medical implant. In some embodiments, the delivery shaft includes a longitudinal lumen. In some embodiments, the microcatheter secures an engagement between the medical implant and the delivery shaft. In some embodiments, the microcatheter provides a conduit for a medical implant to be advanced by the delivery shaft to the treatment location. In many embodiments, a medical system of the present teachings includes an Optical Coherence Tomography (OCT) imaging system. For example, the OCT imaging system can provide visualization of the treatment location, guidance to medical implant deployment, and/or assessment of the deployment status. In some embodiments, the OCT imaging system includes an optical core having a distal end. In some embodiments, the OCT imaging system includes an optical assembly at the distal end of the optical core. In some embodiments, the OCT imaging system includes an elongated shaft where the optical core is placed within an elongated shaft and the optical assembly is positioned at a distal end of the elongated shaft. In some embodiments, the optical assembly is configured to direct light to the surrounding tissue and collect reflected light from the tissue. In some embodiments, the optical core of the OCT imaging system connects optically and mechanically with an interface unit (outside of the body).

In one embodiment of the present teachings, during a medical implant delivery, the microcatheter is slidably disposed over the delivery shaft and the delivery shaft is slidably disposed over the OCT imaging system. In many embodiments, the OCT imaging system is slidably disposed within a central lumen of the radially collapsed medical implant and the longitudinal lumen of the delivery shaft. In one embodiment of the present teachings, the microcatheter, the delivery shaft, and the OCT imaging system extend and retract independently from one another.

According to one embodiment of the present teachings, during a delivery process, the medical implant collapses radially, and the proximal end of the medical implant engages to the distal end of the delivery shaft. In many embodiments, the microcatheter slides over the delivery shaft and the radially collapsed medical implant, securing the engagement between the medical implant and the delivery shaft. In some embodiments, during a delivery, the collapsed medical implant adopts a shape with a reduced diameter to fit inside the microcatheter. In another embodiment, during a deployment process, the microcatheter retracts proximally, exposing the medical implant. In many embodiments, once outside of the microcatheter, the medical implant expands radially and adapts to the diameter of the blood vessel or the shape of the surrounding environment. In many embodiments, upon further proximally withdrawing of the microcatheter and exposing the engagement between the proximal end of the medical implant and the distal end of the delivery shaft, the proximal end of the medical implant is released from the delivery shaft.

In many embodiments, the OCT imaging system of the present teachings is designed for routine cerebrovascular uses. In one embodiment of the present teachings, the OCT imaging system is used prior to implant delivery. In many embodiments, the OCT imaging system uses low-coherence light interferometry. In some embodiments, the OCT imaging system uses near-infrared light, for example, to capture and transmit high-resolution two dimensional (2D) and three dimensional (3D) volumetric structure of the vascular network and other data such as the vessel density and other geometric features, blood flow velocity, flow rate, etc. In many instances, this information allows a physician to make critical treatment decisions prior to delivering a medical implant, for example, to choose the appropriate implant size, adjust the treatment location, etc. In another embodiment, the OCT imaging system is used as a guide for placing a medical implant, for example, with respect to the pathology and arterial wall apposition that are necessary for an effective treatment, as well as a preventive measure for avoiding potential disabling complications. In yet another embodiment, the OCT imaging system is used in a post-implantation assessment. For example, it can allow a surgeon to visualize micron-level features of neurovascular devices as well as assess the effects of the medical implant on the surrounding tissue by visualizing the cerebral arteries' microstructure.

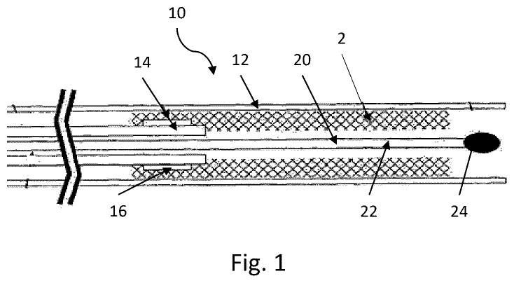

FIG. 1 shows an embodiment of the medical implant (2) being delivered through a microcatheter. As shown in the FIGURE, the medical implant (2) collapses radially with a proximal end of the medical implant (2) engages a distal end of a delivery shaft (14). While the microcatheter (12) remains steady, the delivery shaft (14) and the collapsed medical implant (2) extends into the longitudinal lumen of the microcatheter (12) thereby securing the engagement between the delivery shaft (14) and the medical implant (2). The delivery shaft (14) is configured to push the collapsed medical implant (2) through the longitudinal lumen of the microcatheter (12) toward a distal direction. An OCT imaging system (20) includes an elongated shaft (22) with an optical core disposed therein (not shown) and an optical assembly (24) attached to the distal end of the elongated shaft (22). As shown in the FIGURE, the OCT imaging system (20) extends through a longitudinal lumen of the delivery shaft (14) and a central lumen of the collapsed implant (2), with a distal end portion of the OCT imaging system (20) and the optical assembly (24) extending beyond the distal end of the medical implant (2). In one embodiment of the present teachings, the microcatheter (12), the delivery shaft (14), and the OCT imaging system (20) are configured to extend and retract independently from one another. That is, the distal end of the OCT imaging system (20) can extend distally beyond, and retract proximal to, the distal end of the microcatheter (12) independently; the distal end of the microcatheter can extend distally beyond, and retract proximal to, the distal end of the delivery shaft (14) independently.

As shown in the FIGURE, the exemplary medical implant (2) in the present teachings has a radially collapsed elongated profile (e.g., a reduced diameter and an elongated profile) suitable to be positioned inside and inserted through the microcatheter (12). Once free of the radial constraint, the medical implant expands radially to resume its pre-set radially expanded deployed profile and adapt to the shape of the surrounding environment. In some embodiments, the medical implant is fabricated by laser-cutting or acid-etching a pattern into a preformed tube or sheet, then shape-setting to the intended deployed configuration. In some embodiments, the medical implant is formed from a hollow tube that has been slotted, for example, by using a machining laser or another method, and expands to form an open structure. In another embodiment, the medical implant is formed from wires that are pre-set into the desired shape and then bonded together to connect certain elements on the wires either by cross-hatching, braiding, welding, or other methods of interconnecting rows of wires. In some embodiments, the medical implant is shaped into a tube-like structure. In one embodiment, the wires are welded using a resistance welding technique or an arc welding technique, preferably while in an inert gas environment and with cooling control to control the grain structure in and around the weld site. These joints can be conditioned after the welding procedure to optimize grain sizes to enhance performance.

According to one embodiment of the present teachings, during the implant delivery and/or deployment, the pushing force in the distal direction or the pulling force in the proximal direction are sometimes imposed onto the medical implant (2) along the direction of the longitudinal axis of the microcatheter (14). Thus, the engagement between the medical implant (2) and the delivery shaft (14) is configured to provide a sufficiently strong frictional locking to prevent the medical implant (2) from accidentally disengaging from the delivery shaft (14) prematurely. At the same time, the engagement between the medical implant (2) and delivery shaft (14) is designed to allow an easy relief of the medical implant when the medical implant is free from the constraints imposed by the microcatheter (12). Some embodiments of the engagement between the medical implant and the delivery shaft have been described in the U.S. Pat. No. 11,369,499, filed on Sep. 3, 2014; and U.S. Pat. No. 11,166,827, filed on Mar. 4, 2016; each of which is incorporated by reference herein its entirety.

In one embodiment of the present teachings, an OCT imaging system (20) is used in vivo to provide a real-time cross-sectional visualization of the internal tissue microstructure and/or morphology before, during, and after an implantation of the medical implant. In one embodiment, the OCT imaging system (20) is used to map the blood vessel network, identify aneurysm location, and pinpoint the best implantation location prior to the delivery and deployment of the medical implant. In some embodiments, the OCT imaging system (20) remains inside the vasculature, or adjacent to the implantation location during the deployment process of the medical implant. In another embodiment, the OCT imaging system (20) is used to conduct a post implantation assessment, including assessment of perforating arteries and arterial wall apposition.

Continuing referencing in FIG. 1, the OCT imaging system (20) includes an elongated shaft (22) with an optical core (not shown) positioned within the elongated shaft and an optical assembly (24) at the distal end of the optical core. The optical assembly (24), comprising a lens and a reflector, is constructed to create high-resolution, cross-sectional images of tissue microstructures. Specifically, the lens focuses the light beam onto a sample and the reflector directs the light beam along specific paths within the OCT imaging system. The OCT imaging system measures the optical path length of backscattered light from a sample and compares it to a reference beam. The interference pattern produced by the interaction of these light waves reveals information about the tissue's structure. Continuing referring to FIG. 1, the optical assembly (24) at the distal end of the optical core attaches to a distal end of the elongated shaft (22). In one embodiment of the present teachings, the optical assembly (24) is placed at the distal end of the elongated shaft (22) and faces distally outwards. In another embodiment, the optical assembly (24) is positioned along a tubular surface of the elongated shaft (22) at its distal portion, facing radially away from the elongated shaft (22). In one embodiment, the elongated shaft (22) is configured to rotate around its longitudinal axis, carrying the optical core (not shown) and the optical assembly (24) with it.

In one embodiment, the optical core of the OCT imaging system (20) has two optical fibers, one for transmitting and illuminating the target specimen and the other for collecting the reflected scattered light for reconstruction of the image by the detection system. In another embodiment, the optical core of the OCT imaging system (20) uses a single multi-clad fiber system where the transmission of the source light and the reflected light occurs in a single fiber.

In many embodiments, the OCT imaging system (20) performs in real-time in vivo cross-sectional visualization of the internal tissue microstructure and/or morphology by interferometrically measuring the phase delay of the injected light beam. In one embodiment, the light is delivered from one optical fiber and then split by a fiber coupler into a measurement arm and a reference arm. In many embodiments, the light from the measurement arm propagates through a fiber-based probe and is focused on the tissue surface. In many embodiments, the light beam reflected back from the tissue surface is collected by a probe and passes through the fiber coupler to the receiving fiber arm. In many embodiments, this beam is made to interfere with light reflected from the reference arm. In many embodiments, the interference pattern between these two beams is processed either in the time domain or in the frequency domain. Without being restricted by any theory or hypothesis, the point-wise phase or time delay between the two beams can be quantitatively determined. In one embodiment of the present teachings, to acquire the 3D microstructure morphology of the tissue surface, the optical assembly (24) of the OCT imaging system (20) is configured to rotate around the longitudinal axis of the elongated shaft (22), and extend distally or proximally to scan simultaneously through the tissue surface during the measurement.

According to one embodiment, during a treatment procedure, the OCT imaging system (20) is placed at the treatment location first prior to the delivery and deployment of a medical implant. In many embodiments, the OCT imaging system (20) is advanced through and into the tortuous cerebrovascular anatomy and places the optical assembly (24) at or near the proximity of the treatment location. In many embodiments, the OCT imaging system (20) scans and collects data at the treatment location. Upon analyzing the real-time imaging data, a physician then selects an appropriate type, size, and/or shape of the medical implant, as well as pinpoints the best implantation location. For example, given the information of the real-time vasculature mapping, the physician can also select a medical implant with a relatively smaller radial expansion profile which is suitable for smaller and weaker vessels or a shorter medical implant for a treatment location with a relatively higher density of vessel branches.

Upon selecting an appropriate medical implant as well as deciding on a treatment location, a physician delivers the medical implant to the treatment location. Specifically, a microcatheter (12) is first advanced to the treatment location. As the OCT imaging system (20) remains inside the vasculature, and the optical assembly (24) remains at or near the selected deployment location, the microcatheter (12) tracks over the elongated shaft (22) of the OCT imaging system (20) with a distal end of the microcatheter (12) reaching the treatment location. In one embodiment, the distal end of the microcatheter (12) is placed distally to the treatment location. In another embodiment, the distal end of the microcatheter (12) is placed at or near the treatment location. In one embodiment of the present teachings, the optical assembly (24) of the OCT imaging system (20) remains distally to the distal end of the microcatheter (12) in order to scan and transmit real-time image data for analysis.

With the microcatheter (12) in place, the medical implant (2) can then be delivered. As the OCT imaging system (20) and the microcatheter (12) being held steadily, the medical implant (2) engaging to a delivery shaft (14) at the proximal end of the medical implant (2) is extended over the OCT imaging system (20), and advanced through a longitudinal lumen of the microcatheter (2) to a location approximately to the treatment site.

To deploy the medical implant, the microcatheter (12) is withdrawn proximally to expose the medical implant (2). As the collapsed medical implant (2) is free of the radial constraints imposed by the microcatheter (12), the medical implant (2) self-expands radially until it adapts the shape of the surrounding environment, such as a vessel diameter. During this process, the proximal end of the medical implant (2) remains engaged to the distal end of the delivery shaft (14) and the optical assembly (24) of the OCT imaging system (20) remains distally to the distal end of the medical implant (2).

In one embodiment, during the deployment process, the OCT imaging system (20) is employed to guide the accurate placement of a medical implant (2). For example, before the medical implant (2) is fully released from the delivery shaft (14), the physician can, based on the real-time image scan by the OCT imaging system, reposition the medical implant, slightly distally or proximally, for better treatment. Upon a full radial expansion of portions of the medical implant (2), while the proximal end of the medical implant (2) remains engaged to the delivery shaft (14) and inside the microcatheter (12), the OCT imaging system (20) is employed for a physician to assess the interaction between the medical implant (2) and the surrounding vessel tissue, the post-implantation blood flow condition, and etc. At this point, if needed, the medical implant (2) can be retrieved into the microcatheter (12) by advancing the microcatheter (12) distally while holding the delivery shaft (14) steady, or by retracting the deliver shaft (14) proximally while holding the microcatheter (12) steady, or by a combination of both the advancement of the microcatheter (12) and the retraction of the delivery shaft (14). This movement forces the deployed medical implant (2) back into its radially collapse profile and be constrained back inside the microcatheter (12).

If the physician is satisfied with the partial deployment of the medical implant (2), the medical implant (2) can then be released from the delivery system (10). To do so, the microcatheter (12) is further retracted proximally, freeing the engagement between the proximal end of the medical implant (2) and the distal end of the delivery shaft (14). In one embodiment, the OCT imaging system (20) remains at the treatment location to scan and transmit post-implantation image and data. At the final step of the medical implant deployment process, the delivery system (10), including the microcatheter (12) and the delivery shaft (14), along with the OCT imaging system (20), can then be removed from the patient.

The above-described process provides that the OCT imaging system is placed at the treatment location prior to a microcatheter (12). In an alternative embodiment of the present teachings, the microcatheter (12) is placed prior to the OCT imaging system (20) and the medical implant (2) are placed. Similar to what has been described above, during a treatment procedure, the microcatheter (12) is positioned at or near a treatment location first, creating a conduit for subsequent medical implant delivery. The medical implant (2) engaged to a delivery shaft (14), and the OCT imaging system (20) extending through the longitudinal lumen of the delivery shaft (14) and the central lumen of the collapsed medical implant (2) are advanced together through the microcatheter (12). Once reaching the treatment location, while holding the microcatheter (12) and the medical implant (2) steadily, the physician extends the OCT imaging system (20) distally beyond the distal end of the microcatheter (12) and the distal end of the medical implant (2) and into the vasculature. At this point, the physician can choose the best implantation location as well as select a suitable medical implant. For example, for an aneurysm between two blood vessel branches, a physician could determine an optimized deployment location with a minimum blood flow interruption to the adjacent healthy vessels. Similar to what has been described above, the physician can then deploy, retrieve and/or release the medical implant (2) under the guidance of the real-time image data scanned and transmitted by the OCT imaging system.

Although FIG. 1 of the present teachings illustrates a flow diverter delivered and deployed under the guidance of OCT imaging system, the OCT imaging system can be incorporated with other neurovascular applications, such as the placement of a microstent, a thrombectomy device, an intracranial aneurysm device, a bifurcation aneurysm implant, a vasospasm treatment device, and the like.

Although FIG. 1 of the present teachings illustrates the OCT imaging system extending the longitudinal lumen of the delivery shaft and the central lumen of the flow diverter, the OCT imaging system can extend along with and outside of the delivery shaft and/or the medical implant. For example, for thrombectomy application where the thrombectomy device is joined to a wire (instead of a delivery shaft with a longitudinal lumen), the elongated shaft of the OCT imaging system could extend in a generally parallel direction to the wire, and either through the central lumen of the thrombectomy device or along the outer luminal surface of the device.

FIG. 1 of the present teachings illustrates the delivery shaft detachably engages to the medical implant. A detachable engagement of the present teachings is designed for medical implant intended to remain inside a patient. In the embodiment shown in FIG. 1, a frictional engagement is designed between the proximal end of the medical implant and the distal end of the delivery shaft. Such frictional lock between the medical implant and the delivery shaft should be sufficiently strong to allow axial pushing force and axial pulling force transferred from the delivery shaft to the medical implant, and prevent any accidental disengagement of the medical implant from the delivery shaft before the final release. Various mechanisms can be incorporated to increase frictional locking between the medical implant and the delivery shaft. For example, a friction pad could be incorporated around the exterior luminal surface of the delivery shaft along its distal end portion in order to enhance the frictional lock between the delivery shaft and the medical implant. In another example, the distal end portion of the delivery shaft can incorporate with a surface feature such as shaped grooves or indent, and the proximal end of the medical implant could have a corresponding design feature for a more secure engagement. In yet another embodiment, to secure the engagement between the delivery shaft and the medical implant, additional elements can be incorporated. For example, the delivery system could also include a release tube sliding over the connected proximal end of the medical implant and the distal end of the delivery shaft thereby securing the engagement.

Various embodiments have been illustrated and described herein by way of examples, and one of ordinary skill in the art will appreciate that variations can be made without departing from the spirit and scope of the present teaching. The present teachings are capable of other embodiments or of being practiced or carried out in various other ways. Also, it is to be understood that the phraseology and terminology employed herein is for the purpose of description and should not be regarded as limiting.

Unless otherwise defined, all technical and scientific terms used herein have the same meaning as commonly understood by one of ordinary skill in the art to which the present teachings belong. Methods and materials similar or equivalent to those described herein can be used in the practice or testing of the present teaching. In case of conflict, the patent specification, including definitions, will control. In addition, the materials, methods, and examples are illustrative only and not intended to be limiting.

We claim:

1. A medical system for treating a cerebrovasculature condition, the medical system comprises,
a microcatheter comprising a longitudinal lumen and a distal end;
a medical implant comprising a proximal end, a distal end, and a central lumen, wherein the medical implant has a radially collapsed delivery profile to fit inside the longitudinal lumen of the microcatheter and a radially expanded deployment profile to fit inside the cerebrovasculature;
a delivery shaft comprising a longitudinal lumen and a distal end, wherein the distal end of the delivery shaft detachably engages to a proximal end of the medical implant;
an OCT imaging system comprising an elongated shaft with an optical core and an optical assembly at a distal end of the optical core, wherein the optical core comprises a distal end; wherein the optical assembly attaches to the elongated shaft;
wherein the medical system has a first configuration where the medical implant is slidably disposed within the longitudinal lumen of the microcatheter, the OCT imaging system is slidably disposed within the longitudinal lumen of the delivery shaft and the central lumen of the medical implant, the optical assembly of the OCT imaging system extends distally beyond the distal end of the microcatheter; and
wherein the medical implant has a second configuration where the medical implant is outside of the microcatheter with the optical assembly of the OCT imaging system extending distally beyond the distal end of the medical implant; and
wherein the OCT imaging system, the delivery shaft, and the microcatheter extends and retracts independent from one another.

2. The medical system of claim 1, wherein the medical implant has a braid structure, wherein the braid structure comprises a plurality of filaments intertwined with each other.

3. The medical system of claim 1, wherein the medical implant engages to the delivery shaft when the proximal end of the medical implant remains inside the longitudinal lumen of the microcatheter, and the medical implant detaches from the delivery shaft when the proximal end of the medical implant is outside of the longitudinal lumen of the microcatheter.

4. The medical system of claim 1, wherein the OCT imaging system connects optically and mechanically with an interface unit.

5. The medical system of claim 4, wherein the OCT imaging system is configured to collect and transmit image data of the cerebrovasculature to the interface unit.

6. A method of placing a medical implant inside a cerebrovasculature, the method comprises,
advancing an OCT imaging system into a cerebrovasculature, wherein the OCT imaging system comprises an elongated shaft with an optical core and an optical assembly at a distal end of the optical core and attaching to the elongated shaft;
extending a microcatheter over the elongated shaft of the OCT imaging system and placing a distal end of the microcatheter near a treatment location;
engaging the medical implant to a delivery shaft and advancing the medical implant through an elongated lumen of the microcatheter to the treatment location by extending the medical implant and the delivery shaft over the elongated shaft of the OCT imaging system; and
retracting the microcatheter proximally and allowing the medical implant to expand radially under the guidance of the OCT imaging system.

7. The method of claim 6, comprising collecting and assessing real-time image data of the cerebrovasculature at the treatment location by using the OCT imaging system prior to extending the microcatheter to the treatment location.

8. The method of claim 7, comprising selecting the medical implant based on the real-time image data of the cerebrovasculature.

9. The method of claim 7, comprising selecting a medical implant deployment position based on the real-time image data of the cerebrovasculature.
